(19) United States
(12) Patent Application Publication
(10) Pub. No.: US 2019/0388241 A1
(43) Pub. Date: Dec. 26, 2019

(54) ORTHOPAEDIC SURGICAL INSTRUMENT FOR AN ACETABULAR PROSTHETIC COMPONENT

(71) Applicant: DePuy Synthes Products, Inc., Raynham, MA (US)

(72) Inventors: Michael C. NELSON, Syracuse, IN (US); Darron G. PEDDLE, Millersburg, IN (US)

(21) Appl. No.: 16/018,134

(22) Filed: Jun. 26, 2018

(51) Int. Cl. A61F 2/46; A61F 2/34; A61B 17/16

(52) U.S. Cl. CPC A61F 2/4609; A61F 2/34; A61B 17/1746; A61F 2/4657; A61B 17/1666

(57) ABSTRACT

An orthopaedic surgical instrument includes an insertion tool having a threaded end configured to engage an acetabular cup. A gauge is provided including a pair of clamp arms configured to engage the insertion tool to secure the gauge to the insertion tool. A clamping lever is rotatable to move the clamp arms into engagement with the insertion tool. A visual indicator is configured to indicate a predetermined orientation of the insertion tool relative to a patient's body.

### TECHNICAL FIELD

The present disclosure relates generally to orthopaedic surgical instruments and, more particularly, to surgical instruments used to trial and install an acetabular prosthetic component.

### BACKGROUND

Joint arthroplasty is a well-known surgical procedure by which a diseased and/or damaged natural joint is replaced by a prosthetic joint. For example, in a hip arthroplasty surgical procedure, a patient's natural hip ball and socket joint is partially or totally replaced by a prosthetic hip joint. A typical prosthetic hip joint includes an acetabular prosthetic component and a femoral head prosthetic component. An acetabular prosthetic component generally includes an outer shell configured to engage the acetabulum of the patient and an inner bearing or liner coupled to the shell and configured to engage the femoral head. The femoral head prosthetic component and inner liner of the acetabular component form a ball and socket joint that approximates the natural hip joint.

To facilitate the replacement of the natural joint with a prosthetic hip joint, orthopaedic surgeons may use a variety of orthopaedic surgical instruments such as, for example, reamers, drill guides, drills, positioners, and/or other surgical instruments.

### SUMMARY

According to an aspect of the disclosure, an orthopaedic surgical instrument includes an insertion tool having a threaded end configured to engage an acetabular cup. A gauge is provided including a pair of clamp arms configured to engage the insertion tool to secure the gauge to the insertion tool. Each clamp arm is rotatable about a first axis relative to the insertion tool between a first position in which the clamp arm is spaced apart from the insertion tool and a second position in which the clamp arm engages the insertion tool. A plunger is coupled to the pair of clamp arms. The plunger is moveable along a second axis extending transverse to the first axes to move the clamp arms between the first position and the second position. A clamping lever is provided including a cam that is coupled to the plunger. The clamping lever is rotatable about a third axis extending transverse to the second axis to move the plunger along the second axis. A visual indicator is configured to indicate a predetermined orientation of the insertion tool relative to a patient's body.

In some embodiments, the clamping lever may include a first cam surface spaced a first distance from the third axis, and a second cam surface spaced a second distance from the third axis. The second distance may be greater than the first distance. The clamping lever may include a third cam surface spaced a third distance from the third axis. The third distance may be greater than the second distance.

In some embodiments, the plunger may include a lower wedge configured to engage the clamp arms of the gauge. The lower wedge may include a planar surface extending outward from the second axis. An angled surface may extend outward from the planar surface.

In some embodiments, each clamp arm may include a lower section configured to engage the insertion tool. An upper section may engage the plunger.

In some embodiments, biasing elements may bias each clamp arm into the first position. A biasing element may bias the plunger into a position where each clamp arm can be in the first position.

In some embodiments, the third axis may extend transverse to first axis and second axis.

According to another aspect of the disclosure, a gauge for an orthopaedic surgical instrument includes a pair of clamp arms to secure the gauge to an insertion tool. Each clamp arm is rotatable about a first axis relative to the insertion tool between a first position in which the clamp arm is spaced apart from the insertion tool and a second position in which the clamp arm engages the insertion tool. A plunger is coupled to the pair of clamp arms. The plunger is moveable along a second axis extending transverse to the first axes to move the clamp arms between the first position and the second position. A clamping lever is provided including a cam that is coupled to the plunger. The cam has a plurality of cam surfaces. The clamping lever is rotatable about a third axis extending transverse to the second axis to move the plunger along the second axis. A visual indicator is configured to indicate a predetermined orientation of the insertion tool relative to a patient's body.

In some embodiments, the plurality of cam surfaces may include a first cam surface spaced a first distance from the third axis. A second cam surface may be spaced a second distance from the third axis. The second distance may be greater than the first distance. The plurality of cam surfaces may include a third cam surface spaced a third distance from the third axis. The third distance may be greater than the second distance.

In some embodiments, the plunger may include a lower wedge configured to engage the clamp arms of the gauge. The lower wedge may include a planar surface extending outward from the second axis. An angled surface may extend outward from the planar surface.

In some embodiments, each clamp arm may include a lower section configured to engage the insertion tool. An upper section may engage the plunger.

In some embodiments, biasing elements may bias each clamp arm into the first position. A biasing element may bias the plunger into a position where each clamp arm can be in the first position.

In some embodiments, the third axis may extend transverse to first axis and second axis.

According to yet another aspect of the disclosure, a method of securing a gauge to an insertion tool includes positioning a pair of clamp arms of the gauge around a shaft of the insertion tool. The method also includes rotating a clamping lever so that a first cam surface engages a plunger that moves to rotate the clamp arms into a first position around the shaft. The method also includes rotating the clamping lever so that a second cam surface engages the plunger and moves the plunger to rotate the clamp arms into a second position, wherein the clamp arms engage the shaft. The method also includes rotating the clamping lever so that a third cam surface engages the plunger and moves the plunger to rotate the clamp arms into a third position, wherein the clamp arms are secured to the shaft.

In some embodiments, each clamp arm may be rotatable about a first axis relative to the insertion tool. The plunger may be moveable along a second axis extending transverse to the first axes. The clamping lever may be rotatable about a third axis extending transverse to the second axis.

### BRIEF DESCRIPTION OF THE DRAWINGS

The detailed description particularly refers to the following figures, in which:

FIG. 1 is a perspective view of an orthopaedic surgical instrument including a gauge;

FIG. 2 is a perspective view of the gauge shown in FIG. 1;

FIG. 3 is an exploded elevation view of the gauge shown in FIG. 1;

FIG. 4 is a perspective view of a housing of the gauge shown in FIGS. 1-3;

FIG. 5 is a rear elevation view of the housing shown in FIG. 4;

FIG. 6 is a partial side elevation view of a clamping lever of the gauge shown in FIGS. 1-3;

FIG. 7 is a rear perspective view of the clamping lever shown in FIG. 6;

FIG. 8 is a side perspective view of a plunger of the gauge shown in FIGS. 1-3;

FIG. 9 is a cross-sectional elevation view of the gauge along line 9-9 in FIG. 1 showing the gauge in a detached position;

FIG. 10 is a cross-sectional elevation view of the gauge showing the gauge in the detached position taken along line 10-10 in FIG. 1;

FIG. 11 is a cross-sectional elevation view similar to FIG. 9 showing the gauge in an initial clamped position;

FIG. 12 is a cross-sectional elevation view similar to FIG. 10 showing the gauge in the initial clamped position of FIG. 11;

FIG. 13 is a cross-sectional elevation view similar to FIGS. 9 and 11 showing the gauge in another clamped position; and

FIG. 14 is a cross-sectional elevation view of the gauge taken along the line 14-14 in FIG. 1 and showing the gauge in the clamped position of FIG. 13.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

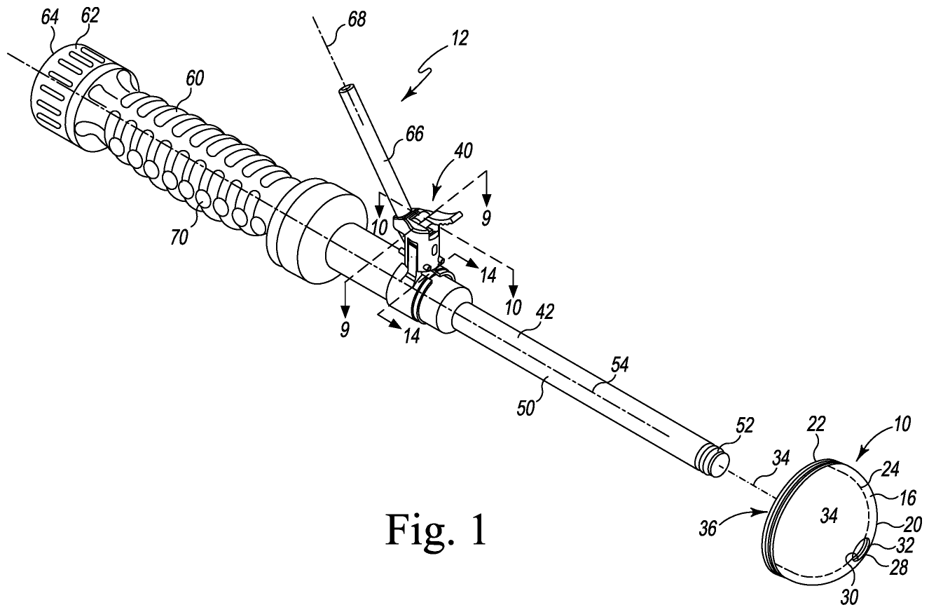

Referring now to FIG. 1, an acetabular prosthetic component 10 and an orthopaedic surgical instrument 12 (hereinafter instrument 12) are shown. The instrument 12 may be used during a hip arthroplasty surgical procedure to trial and install the acetabular prosthetic component 10 in a patient's pelvic bone. It should be appreciated, however, that although the instrument 12 is described below in regard to the performance of a hip arthroplasty surgical procedure, certain concepts associated with the instrument 12 may be utilized in replacement procedures of numerous other joints throughout the body. In other words, one or more of the elements of the instrument 12 may be incorporated into surgical instruments used in, for example, knee, spinal, shoulder, or other replacement procedures.

The acetabular prosthetic component 10 includes an acetabular shell component 16 configured to be implanted within a surgically-prepared acetabulum of the patient's pelvic bone. The acetabular shell component 16 includes an outer surface 20 having a convex shape that is hemispherical or partially spherical. The acetabular shell component 16 also includes an annular face 22 and an inner surface 24 having a concave shape that is partially spherical in shape extends inwardly from the face 22. The inner surface 24 defines a cavity 26 in the acetabular shell component 16 that is sized to receive a metallic or polymeric insert.

The acetabular shell component 16 has a passageway 28 defined therein. The passageway 28 is defined by a cylindrical inner wall 30. As shown in FIG. 1, the inner wall 30 has a plurality of internal threads 32 defined thereon.

The acetabular prosthetic component 10 has an acetabular axis 34 that extends outwardly from the acetabular shell component 16. The acetabular axis 34 extends through the center 36 of the annular face 22 and the apex of the inner surface 24. The acetabular axis 34 is oriented at a desired abduction or inclination angle and a desired anteversion angle when the acetabular prosthetic component 10 is properly positioned in the patient's surgically-prepared acetabulum.

The instrument 12 includes a gauge 40 and an insertion tool 42 that may be secured to the acetabular prosthetic component 10. When the insertion tool 42 is attached to the gauge 40 with the prosthetic component 10, the surgeon may use the gauge 40 to guide the insertion of the acetabular prosthetic component 10 into the patient's surgically prepared acetabulum and assist the surgeon in orienting the acetabular prosthetic component 10 at the desired abduction or inclination angle and the desired anteversion angle.

The insertion tool 42 includes a shaft 50 having a threaded end 52. The threaded end 52 is configured to couple the internal threads 32 of the acetabular prosthetic component 10. The shaft 50 extends along a longitudinal axis 54 that is collinear with the acetabular axis 34 when the acetabular prosthetic component 10 is coupled to the shaft 50. A handle 60 extends from the shaft 50 along the longitudinal axis 54. An end 62 of the handle 60 includes an impaction plate 64 that may be struck by the surgeon to implant the acetabular prosthetic component 10 in the surgically-prepared acetabulum. The handle 60 also includes a grip 70 secured to the handle 60. The grip 70 includes a knurled outer surface 72 that is sized to receive the hand of a surgeon or other user. It should be appreciated that in other embodiments the outer surface may be substantially smooth or include a number of ribs to support the hand of a user.

The gauge 40 includes a visual indicator 66 that aligns the insertion tool 42 for proper placement of the acetabular prosthetic component 10. The visual indicator 66 extends along a longitudinal axis 68 angled obliquely to the longitudinal axis 54 of the insertion tool 42. In the illustrative embodiment, the longitudinal axis 68 is angled approximately 45 degrees with respect to the longitudinal axis 54. In some embodiments, the longitudinal axis 68 is angled within a range of 35 degrees to 45 degrees with respect to the longitudinal axis 54. During insertion of the acetabular prosthetic component 10 into the surgically-prepared acetabulum, the instrument 12 may be used to indicate an acceptable level of acetabular shell inclination. Once assembled, the instrument 12 should be raised until the visual indicator 66 is perpendicular to the plane of the operating table with the patient in the lateral decubitus position. The instrument 12 is rotated so that the longitudinal axis 68 of the visual indicator 66 is aligned perpendicular to the coronal plane of the patient and parallel to the transverse plane and sagittal plane of the patient. As described in greater detail below, the gauge 40 includes a locking mechanism 74 that is configured to lock the gauge 40 in position relative to the shaft 50 of the insertion tool 42 and make the gauge 40 stable under impaction forces applied to the tool 42 to implant the acetabular prosthetic component 10.

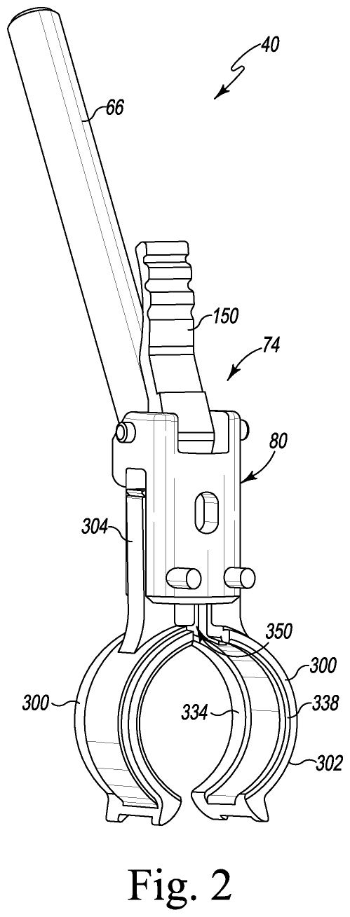

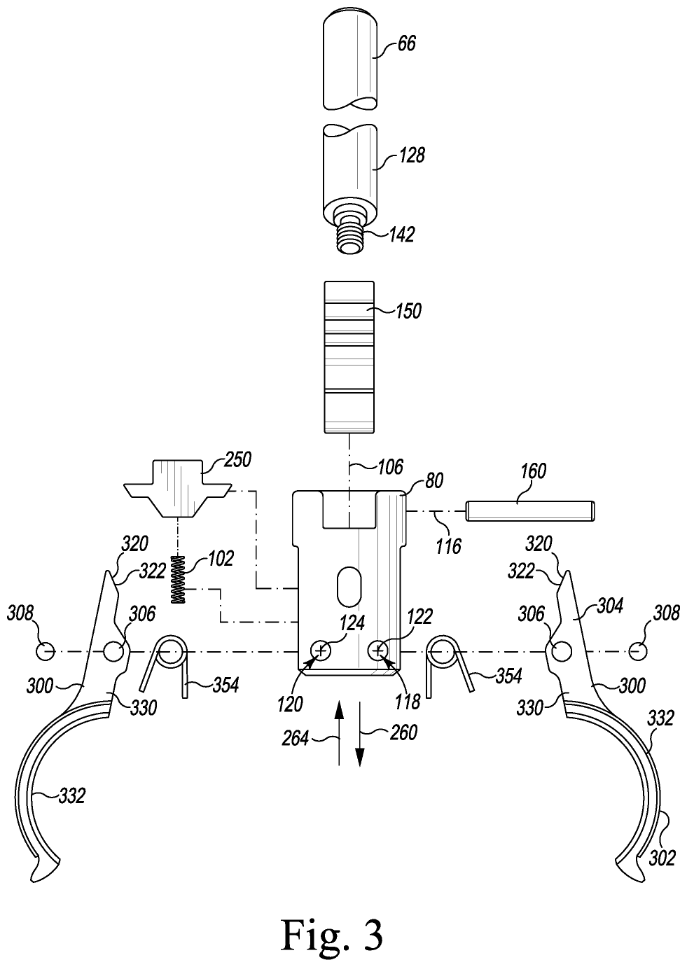

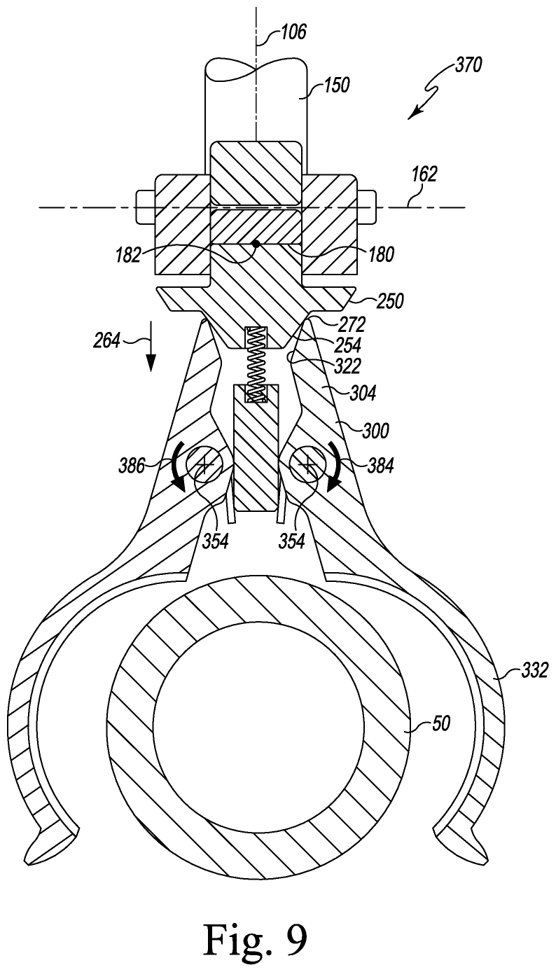

Referring now to FIGS. 2-3, the locking mechanism 74 is coupled to a housing 80 of the gauge 40. In the illustrative embodiment, the locking mechanism 74 includes a pair of clamp arms 300 that are pivotally coupled to the housing 80 and a clamping lever 150 that is pivotally coupled to the housing 80. The arms 300 extend downwardly away from the housing 80, while the clamping lever 150 extends upwardly from the housing 80 adjacent to the visual indicator 66. As described in greater detail below, the clamping lever 150 is configured to act on a plunger 250 positioned in the housing 80 to cause the clamp arms 300 to move relative to the housing 80 between an unclamped position (see FIG. 9) and a number of clamped positions (see FIGS. 12-14). In that way, the locking mechanism 74 has multiple clamped or locked positions such that if the insertion tool 42 or gauge 40 wears over extended use, the locking mechanism 74 will continue to have sufficient locking positions to secure to the gauge 40 to the tool 42.

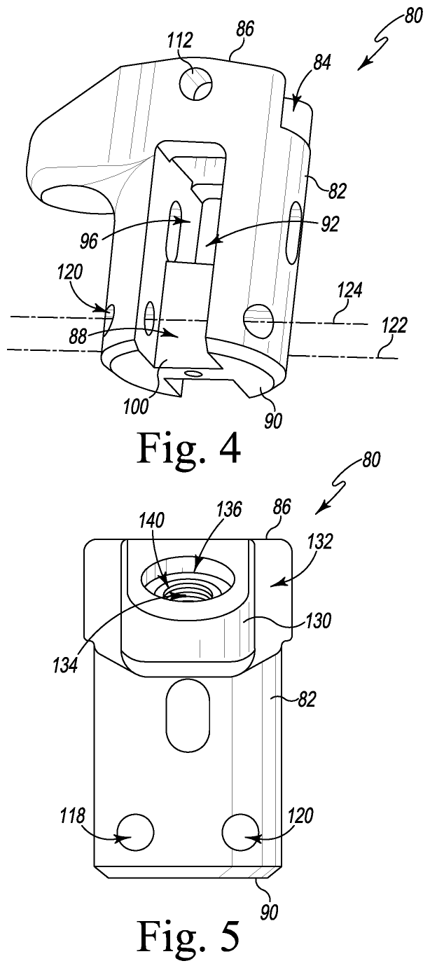

Referring now to FIGS. 4-5, the housing 80 of the gauge 40 includes a body 82 extending from a top end 86 to a bottom end 90. An opening 84 sized to receive a portion of the clamping lever 150 is defined in the top end 86. The housing 80 also includes a pair of channels 88, 92 that extend inwardly from the bottom end 90. In the illustrative embodiment, the opening 84 and the upper ends of the channels 88, 92 open into a cavity 96 defined in the body 82. As shown in FIG. 4, the body 82 includes a support beam 100 that extends from the bottom end 90 and separates the lower ends of the channels 88, 92.

In the illustrative embodiment, the clamping lever 150 and the clamp arms 300 are coupled to the housing 80 via a number of elongated pins 160, 308 (see FIG. 3), and the housing 80 includes a number of pin holes 112, 118, 120 sized to receive the respective the pins 160, 308, respectively. The pin holes 112 extend along an axis 116 and open into the opening 84 defined in the top end 86 of the body 82. The holes 112 cooperate to receive the elongated pin 160 to pivotally couple the clamping lever 150 to the housing 80.

The pin holes 118 extend along an axis 122 and open into the channel 88 on one side of the support beam 100. The holes 118 cooperate to receive one of the elongated pins 308 to pivotally couple one of the clamp arms 300 to the housing 80. The other pin holes 120 extend along an axis 124 extending parallel to the axis 122 and open into the channel 92 on the other side of the support beam 100. The holes 118 cooperate to receive the other elongated pin 308 to pivotally couple the other clamp arm 300 to the housing 80. It should be appreciated that in other embodiments the clamp arm 300 may include pins to couple the clamp arm 300 to the housing 80. As shown in FIG. 4, the axes 122, 124 extend transverse (and orthogonal to) the axis 116 such that the clamp lever 150 and the clamp arms 300 pivot in different directions. The axes 116, 122, 124 also extend transverse (and orthogonal to) the axis 106.

As described above, the gauge 40 also includes the visual indicator 66, which is configured to be secured to the housing 80. In the illustrative embodiment, the visual indicator 66 includes an elongated rod 128 configured to engage the housing body 82. As shown in FIG. 5, the housing 80 includes a flange 130 that extends from the rear 132 of the upper end 96 of the body 82. An opening 136 is formed in the flange 130, and a threaded inner surface 140 extends into the flange 130 to define a threaded bore 134. As shown in FIG. 3, the elongated rod 128 includes a threaded end 142 that is sized to be positioned in the threaded bore 134 to couple the visual indicator 66 to the housing 80. It should be appreciated that in other embodiments the visual indicator 66 may be press fit into the housing 80, formed integrally with the housing 80 of otherwise coupled to the housing 80.

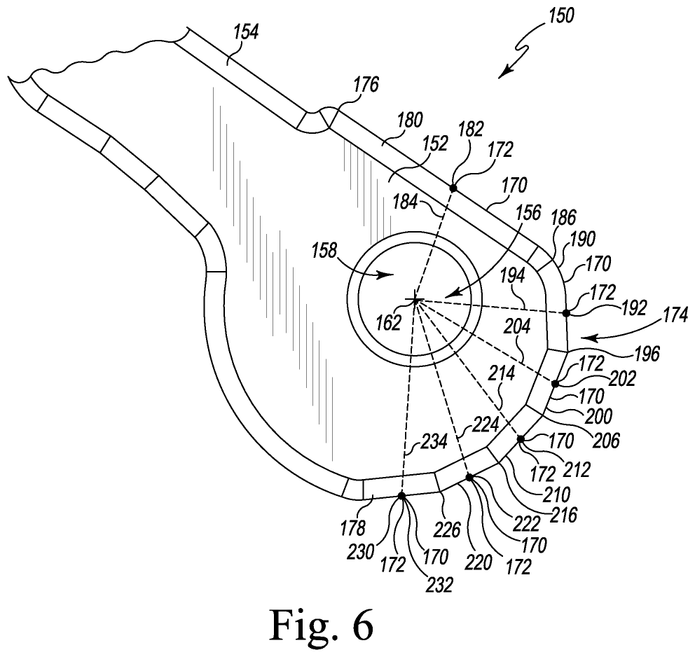

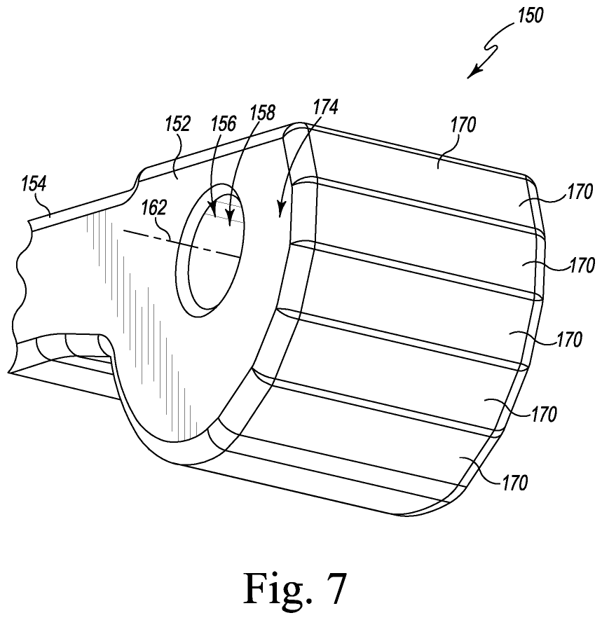

As described above, the locking mechanism 74 includes a clamping lever 150 that is pivotally coupled to the housing 80 via the elongated pin 160. Referring now to FIGS. 6-7, the clamping lever 150 includes a base 152 and a lever arm 154 extending from the base 152 to an outer tip. The lever arm 154 is configured to be actuated by the surgeon to couple the gauge 40 to the insertion tool 42, as described in greater detail below. The base 152 includes a passageway 156 that extends between openings 158 and is sized to receive the pin 160. In that way, the clamping lever 150 is configured to rotate about the axis 116 defined by the pin holes 112.

The base 152 includes a plurality of planar cam surfaces 170 that define a cam 174 configured to engage the plunger 250. As shown in FIG. 6, the cam 174 extends from an edge 176 of one surface 170 to another edge of another surface 178. Each cam surface 170 has a midpoint 172, and a different distance is defined between the midpoint 172 of each surface 170 and an axis 162, as shown in FIG. 6. In the illustrative embodiment, the distances increase when moving in a clockwise direction around the axis 162 from the cam edge 176 to the cam edge 178.

As shown in FIG. 6, a cam surface 180 extending from edge 176 has a midpoint 182 between the edge 176 and an edge 186 that is spaced a distance 184 from the axis 162. A cam surface 190 connected to the cam surface 180 has a midpoint 192 between the edge 186 and an edge 196 that is spaced a distance 194 from the axis 162 that is greater than the distance 184. A cam surface 200 connected to the cam surface 190 has a midpoint 202 between the edge 196 and an edge 206 that is spaced a distance 204 from the axis 162 that is greater than the distance 194. A cam surface 210 connected to the cam surface 200 has a midpoint 212 between the edge 206 and an edge 216 that is spaced a distance 214 from the axis 162 that is greater than the distance 204. A cam surface 220 connected to the cam surface 210 has a midpoint 222 between the edge 216 and an edge 226 that is spaced a distance 224 from the axis 162 that is greater than the distance 214. A cam surface 230 connected at one end to the cam surface 220 and at the other end to edge 178 has a midpoint 232 between the edge 226 and the edge 178 that is spaced a distance 234 from the axis 162 that is greater than the distance 224.

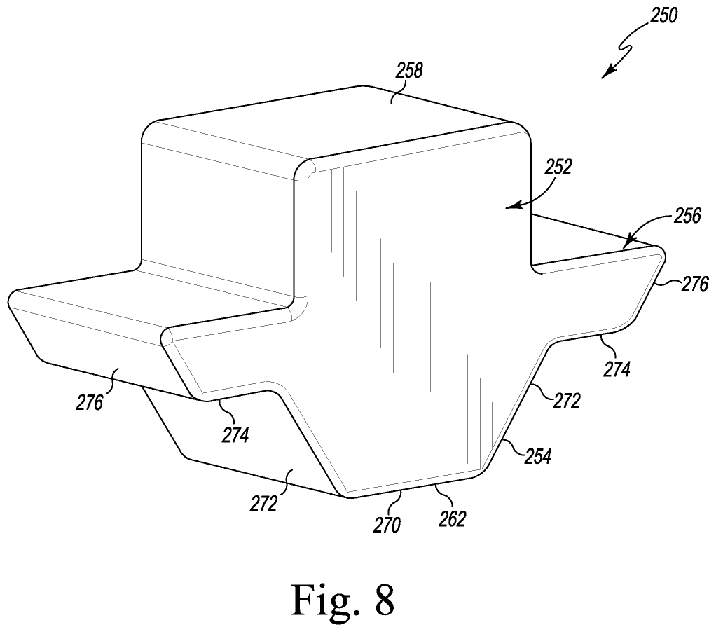

As described above, the cam surfaces 170 are configured to separately engage the plunger 250 positioned in the housing 80. Referring now to FIG. 8, the plunger 250 includes a body 252 having a lower wedge 254 and an upper wedge 256. A top 258 of the plunger 250 engages the cam surfaces 170 so that the cam 152 moves the plunger 250 along the axis 106. As the distance of the cam surface 170 from the axis 162 increases, the plunger 250 is biased in a first direction 260. An end 262 of the lower wedge 254 engages the biasing element 102 to bias the plunger 250 along the axis 106 in a second direction 264 that is opposite the first direction 260.

The lower wedge 254 includes the end 262, which includes a planar surface 270 that extends between a pair of angled surfaces 272. The upper wedge 256 includes planar surfaces 274 that extend outward from the body 252. An angled surface 276 extends outward from each planar surface 274. The end 262 of the lower wedge 254 engages the biasing element 102. The biasing element 102 is secured between the plunger 250 and the support beam 100 to bias the plunger 250 along the axis 106 in the second direction 264.

Returning to FIGS. 2-3 each clamp arm 300 includes a lower section 302 that is configured to engage the insertion tool 42, and an upper section 304 that engages the plunger 250. The upper section 304 includes a pair of pin holes 306 to receive the pin 308 to secure each clamp arm 300 to the housing 80. An end 320 of the upper section 304 includes an angled surface 322 that engages the surfaces 272, 274, and 276 of the plunger 250 so that when the plunger 250 is biased in the first direction 260, the surfaces 272, 274, and 276 bias the upper section 304 of each clamp arm 300 outward, thereby moving the lower section 302 of each clamp arm 300 inward.

Each lower section 302 includes a straight arm 330 and an arcuate arm 332. The arcuate arm 332 is sized and shaped to be positioned around the shaft 50 of the insertion tool 42. Each arcuate arm 332 includes a large rib 334 extending along an inner surface 336, and a small rib 338 extending along the inner surface 336. The ribs 334 and 338 are configured to engage grooves of the shaft 50, when the gauge 40 is coupled to the insertion tool 42, as described in more detail below. Each lower section 302 also includes a slot 350 formed on an inner surface 352 of the straight arm 330. A biasing element 354 is positioned within the slot 350 and engages the support beam 100 of the housing 80. In the illustrative embodiment, the biasing element 354 is a torsion spring. The biasing element 354 biases the clamp arms into an open position, wherein the lower sections 302 of the clamp arms 300 are spaced apart from the shaft 50 of the insertion tool 42.

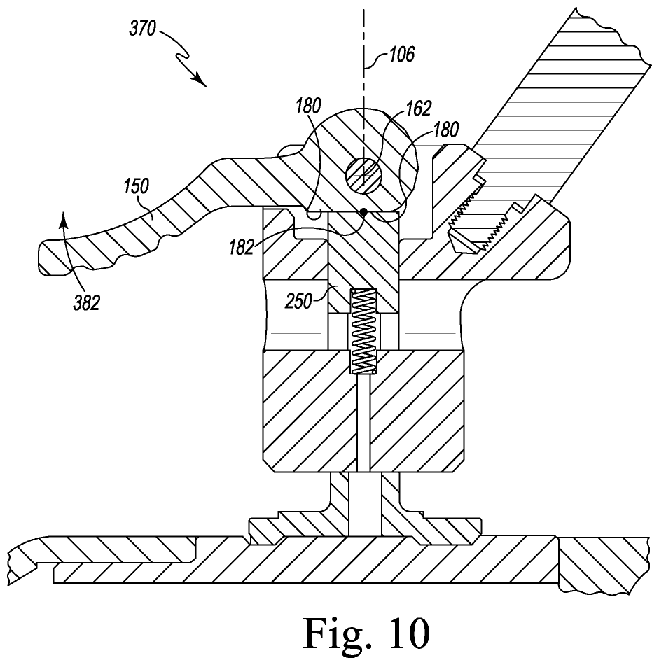

Referring now to FIGS. 9-14, the gauge 40 and its locking mechanism 74 are shown in a number of positions relative to the insertion tool 42. Referring to FIGS. 9-10, the gauge 40 is illustrated in an open position 370. In the open position 370, the clamping lever 150 is rotated so that the cam surface 180 is positioned against the plunger 250, as illustrated in FIG. 10. In this position, the distance 184 from the midpoint 182 enables the biasing element 102 to translate the plunger 250 along the axis 106 toward the axis 116. As illustrated in FIG. 10, the lower wedge 254 of the plunger 250 engages the upper section 304 of each clamp arm 300, in the open position 370. The angled surfaces 322 at the end 320 of the upper section 304 of each clamp arm 300 engages the angled surfaces 272 of the plunger 250 so that the lower section 302 of each clamp arm 300 is biased outward by the biasing elements 354. The clamp arms 300 rotate about the axes 310 to move the arcuate arms 332 away from the shaft 50 in the open position 370.

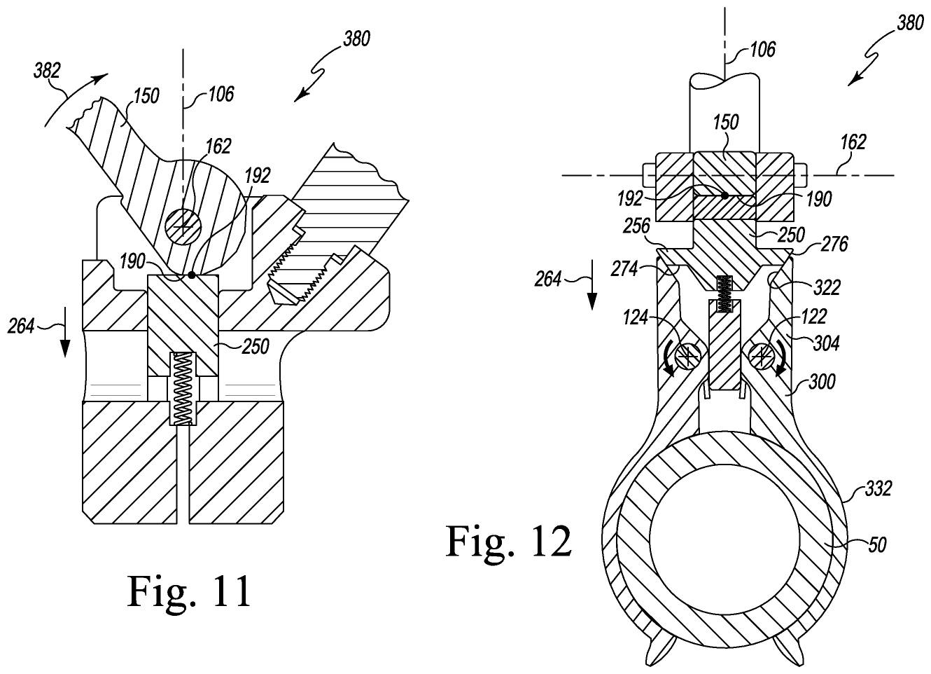

In FIGS. 11-12, the gauge 40 is illustrated in an intermediate position 380, wherein the clamping lever 150 is rotated about arrow 382 so that the cam surface 190 engages the plunger 250. Because the distance 194 from midpoint 192 is greater than the distance 184 from midpoint 182, the biasing element 102 is compressed and the plunger 250 is translated in the first direction 260 along the axis 106 toward the support beam 100. As the plunger 250 translates along the axis 106, the angled surfaces 322 at the end 320 of the upper section 304 of each clamp arm 300 move along the planar surfaces 274 of the upper wedge 256 and into engagement with the angled surfaces 276, as illustrated in FIG. 12. In the intermediate position 380, the clamp arms 300 rotate about the axes 122, 124 in the direction of arrows 384, 386 so that the arcuate arms 332 move inward and engage the shaft 50.

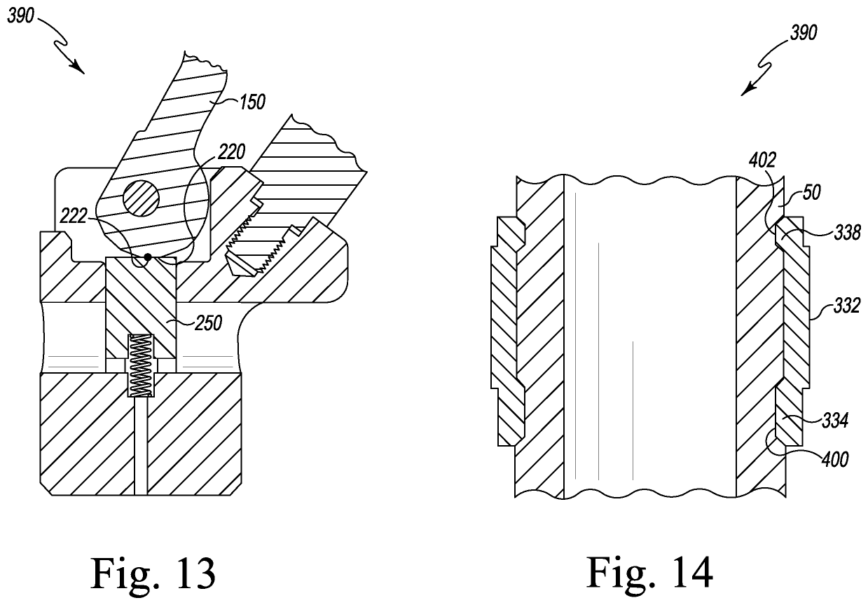

Referring to FIGS. 13-14, the gauge 40 is illustrated in an engaged position 390. The clamping lever 150 is rotated about arrow 382 such that the cam surface 220 engages the plunger 250. Because the distance 224 from midpoint 222 is greater than the distance 194 from midpoint 192, the biasing element 102 is further compressed and the plunger 250 is locked into the engaged position 390. As illustrated in FIG. 14, arcuate arms 332 of the clamp arms 300 are secured against the shaft 50. In the engaged position 390, the large rib 334 is secured within a large groove 400 formed in the shaft 50, and the small rib 338 is secured within a small groove 402 formed in the shaft 50. In some embodiments, the arcuate arms 332 do not include the large rib 334 and the small rib 338, and the shaft 50 does not include the grooves 400 and 402. In such an embodiment, the arcuate arms 332 are secured to the shaft 50 by friction.

To release the gauge 40 from the shaft 50, the clamping lever 150 is rotated in the direction opposite arrow 382 so that the cam 174 clocks through the cam surfaces 170 to the cam surface 180, thereby releasing the compression on the biasing element 102. As the biasing element 102 is expanded, the plunger 250 moves in the second direction 264 so that the upper section 304 of each clamp arm 300 moves to the lower wedge 254 of the plunger 250 and the arcuate arms 332 rotate in the direction opposite arrows 384, 386 to disengage the large rib 334 and small rib 338 from the shaft 50 into the open position 370.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the devices and assemblies described herein. It will be noted that alternative embodiments of the devices and assemblies of the present disclosure may not include all of the features described yet still benefit from at least some of the advantages of such features. Those of ordinary skill in the art may readily devise their own implementations of the devices and assemblies that incorporate one or more of the features of the present invention and fall within the spirit and scope of the present disclosure as defined by the appended claims.

1. An orthopaedic surgical instrument comprising:
an insertion tool having a threaded end configured to engage an acetabular cup,
a gauge including a pair of clamp arms configured to engage the insertion tool to secure the gauge to the insertion tool, each clamp arm being rotatable about a first axis relative to the insertion tool between a first position in which the clamp arm is spaced apart from the insertion tool and a second position in which the clamp arm engages the insertion tool,
a plunger coupled to the pair of clamp arms, the plunger being moveable along a second axis extending transverse to the first axes to move the clamp arms between the first position and the second position,
a clamping lever including a cam that is coupled to the plunger, the clamping lever being rotatable about a third axis extending transverse to the second axis to move the plunger along the second axis, and
a visual indicator configured to indicate a predetermined orientation of the insertion tool relative to a patient's body.

2. The orthopaedic surgical instrument of claim 1, wherein the clamping lever further comprises:
a first cam surface spaced a first distance from the third axis, and
a second cam surface spaced a second distance from the third axis, wherein the second distance is greater than the first distance.

3. The orthopaedic surgical instrument of claim 2, wherein the clamping lever further comprises a third cam surface spaced a third distance from the third axis, wherein the third distance is greater than the second distance.

4. The orthopaedic surgical instrument of claim 1, wherein the plunger further comprises a lower wedge configured to engage the clamp arms of the gauge.

5. The orthopaedic surgical instrument of claim 5, wherein the lower wedge further comprises:
a planar surface extending outward from the second axis, and
an angled surface extending outward from the planar surface.

6. The orthopaedic surgical instrument of claim 1, wherein each clamp arm further comprises:
a lower section configured to engage the insertion tool, and
an upper section that engages the plunger.

7. The orthopaedic surgical instrument of claim 1, further comprising biasing elements to bias each clamp arm into the first position.

8. The orthopaedic surgical instrument of claim 1, further comprising a biasing element to bias the plunger into a position where each clamp arm can be in the first position.

9. The orthopaedic surgical instrument of claim 1, wherein the third axis extends transverse to first axis and second axis.

10. A gauge for an orthopaedic surgical instrument, the gauge comprising:
a pair of clamp arms to secure the gauge to an insertion tool, each clamp arm being rotatable about a first axis relative to the insertion tool between a first position in which the clamp arm is spaced apart from the insertion tool and a second position in which the clamp arm engages the insertion tool,
a plunger coupled to the pair of clamp arms, the plunger being moveable along a second axis extending transverse to the first axes to move the clamp arms between the first position and the second position,
a clamping lever including a cam that is coupled to the plunger, the cam having a plurality of cam surfaces, the clamping lever being rotatable about a third axis extending transverse to the second axis to move the plunger along the second axis, and
a visual indicator configured to indicate a predetermined orientation of the insertion tool relative to a patient's body.

11. The gauge of claim 10, wherein the plurality of cam surfaces further comprises:
a first cam surface spaced a first distance from the third axis, and
a second cam surface spaced a second distance from the third axis, wherein the second distance is greater than the first distance.

12. The gauge of claim 11, wherein the plurality of cam surfaces further comprises a third cam surface spaced a third distance from the third axis, wherein the third distance is greater than the second distance.

13. The gauge of claim 10, wherein the plunger further comprises a lower wedge configured to engage the clamp arms of the gauge.

14. The gauge of claim 13, wherein the lower wedge further comprises:
a planar surface extending outward from the second axis, and
an angled surface extending outward from the planar surface.

15. The gauge of claim 10, wherein each clamp arm further comprises:
a lower section configured to engage the insertion tool, and
an upper section that engages the plunger.

16. The gauge of claim 10, further comprising biasing elements to bias each clamp arm into the first position.

17. The gauge of claim 10, further comprising a biasing element to bias the plunger into a position where each clamp arm can be in the first position.

18. The gauge of claim 10, wherein the third axis extends transverse to first axis and second axis.

19. A method of securing a gauge to an insertion tool comprising:
positioning a pair of clamp arms of the gauge around a shaft of the insertion tool,
rotating a clamping lever so that a first cam surface engages a plunger that moves to rotate the clamp arms into a first position around the shaft,
rotating the clamping lever so that a second cam surface engages the plunger and moves the plunger to rotate the clamp arms into a second position, wherein the clamp arms engage the shaft, and
rotating the clamping lever so that a third cam surface engages the plunger and moves the plunger to rotate the clamp arms into a third position, wherein the clamp arms are secured to the shaft.

20. The method of claim 19, wherein:
each clamp arm is rotatable about a first axis relative to the insertion tool,
the plunger is moveable along a second axis extending transverse to the first axes, and
the clamping lever is rotatable about a third axis extending transverse to the second axis.
